(19) United States
(12) Patent Application Publication
(10) Pub. No.: US 2024/0142882 A1
(43) Pub. Date: May 2, 2024

(54) SYSTEM AND METHOD FOR DISCOVERING PHOTORESIST DISSOLVENT

(71) Applicant: SAMSUNG ELECTRONICS CO., LTD., Suwon-si (KR)

(72) Inventors: Thanh Cuong NGUYEN, Suwon-si (KR); Jiyoung PARK, Suwon-si (KR); Jungah Kim, Suwon-si (KR); Seungmin LEE, Suwon-si (KR); Inkook JANG, Suwon-si (KR)

(73) Assignee: SAMSUNG ELECTRONICS CO., LTD., Suwon-si (KR)

(21) Appl. No.: 18/382,349

(22) Filed: Oct. 20, 2023

(30) Foreign Application Priority Data
Oct. 26, 2022 (KR) 10-2022-0139625

(51) Int. Cl. G03F 7/00; G03F 7/40

(52) U.S. Cl. CPC G03F 7/70525; G03F 7/40; H01L 21/0274

(57) ABSTRACT

A method for discovering a new photoresist dissolvent includes obtaining input data defining a ligand material, estimating a reaction energy of a ligand exchange reaction in which a first ligand of a first complex including a first metal and the first ligand is exchanged with a second ligand, based on the input data, estimating a residual concentration of the first metal corresponding to the reaction energy based on a physical model, and verifying a photoresist dissolvent providing the second ligand based on the residual concentration.

### CROSS-REFERENCE TO RELATED APPLICATION

This application is based on and claims priority under 35 USC § 119 to Korean Patent Application No. 10-2022-0139625, filed on Oct. 26, 2022, in the Korean Intellectual Property Office, the disclosure of which is incorporated by reference herein in its entirety.

### BACKGROUND

The present disclosure relates to a photoresist dissolvent, and more specifically, to a system and method for discovering the photoresist dissolvent.

Photolithography refers to a process of forming a pattern by transferring a geometric pattern of a photomask to a photosensitive chemical photoresist by using light. After the pattern is formed, the photoresist may be removed from the pattern by a photoresist dissolvent. A shorter wavelength light may be used for forming a finer pattern, and thus a new photoresist and photoresist dissolvent may be required.

### SUMMARY

Example embodiments provide a system and method for discovering a new photoresist dissolvent by verifying the photoresist dissolvent.

According to an aspect of an example embodiment, a method includes: obtaining input data defining a ligand material; estimating a reaction energy of a ligand exchange reaction based on the input data, a first ligand of a first complex including a first metal and the first ligand being exchanged with a second ligand in the ligand exchange reaction; estimating a residual concentration of the first metal corresponding to the reaction energy, based on a physical model; and verifying a photoresist dissolvent providing the second ligand based on the residual concentration.

According to an aspect of an example embodiment, a system including: a non-transitory storage medium storing instructions; and at least one processor configured to execute the instructions to: obtain input data defining a ligand material; estimate a reaction energy of a ligand exchange reaction based on the input data, a first ligand of a first complex including a first metal and the first ligand being exchanged with a second ligand in the ligand exchange reaction; estimate a residual concentration of the first metal corresponding to the reaction energy based on a physical model; and verify a photoresist dissolvent providing the second ligand based on the residual concentration.

According to an aspect of an example embodiment, a non-transitory storage medium storing instructions executed by at least one processor to perform a method including: obtaining input data defining a ligand material; estimating a reaction energy of a ligand exchange reaction based on the input data, a first ligand of a first complex including a first metal and the first ligand being exchanged with a second ligand in the ligand exchange reaction; estimating a residual concentration of the first metal corresponding to the reaction energy based on a physical model; and verifying a photoresist dissolvent providing the second ligand based on the residual concentration.

### BRIEF DESCRIPTION OF THE DRAWINGS

The above and other aspects, features, and advantages of certain example embodiments of the present disclosure will be more apparent from the following detailed description taken in conjunction with the accompanying drawings in which:

FIG. 1 is a flowchart showing a method of discovering a photoresist dissolvent, according to an embodiment;

FIGS. 2A and 2B are diagrams illustrating processing steps for manufacturing an integrated circuit in the semiconductor manufacturing process according to an embodiment;

FIG. 3 is a diagram illustrating an operation involving the photoresist dissolvent according to an embodiment;

FIG. 4 is a reaction scheme illustrating a ligand exchange reaction model according to an embodiment;

FIGS. 5A and 5B are tables showing examples of input data IN according to an embodiment;

FIG. 6 is a flowchart illustrating an aspect of a method of discovering a photoresist dissolvent, according to an embodiment;

FIG. 7 is a graph illustrating a physical model PM according to an embodiment;

FIG. 8 is a flowchart showing another aspect of a method of discovering a photoresist dissolvent according to an embodiment;

FIG. 9 is a flowchart showing still another aspect of a method of discovering a photoresist dissolvent according to an embodiment;

FIG. 10 is a graph illustrating an example of the concentration range defined by the threshold value, according to an embodiment; and

FIG. 11 is a block diagram showing a computing system 100, according to an embodiment.

### DETAILED DESCRIPTION

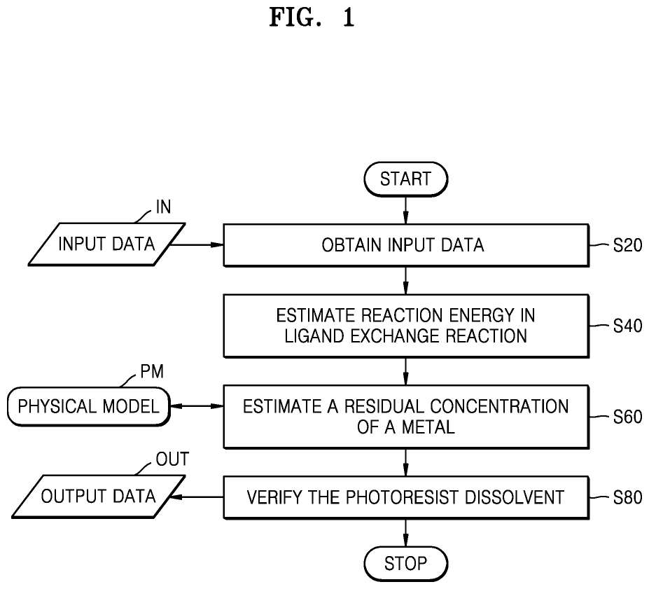

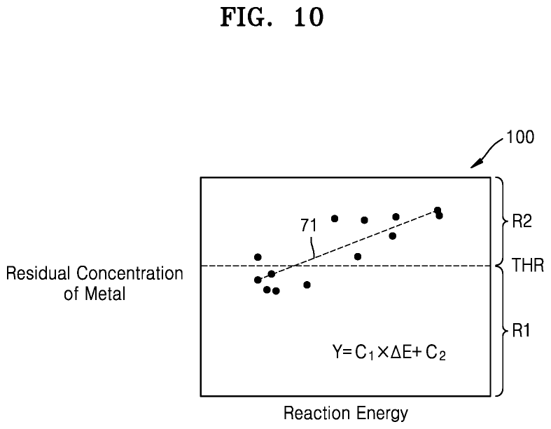

FIG. 1 is a flowchart showing a method of discovering a photoresist dissolvent, according to an embodiment. Hereinafter, a method of discovering a photoresist dissolvent may be referred to as a method of verifying a photoresist dissolvent. The method shown in FIG. 1 may be performed in any computing system (e.g., 100 in FIG. 10). As shown in FIG. 1, the method of discovering a photoresist dissolvent may include a plurality of operations S20, S40, S60, and S80.

Various operations of the exemplary methods described below may be performed by any suitable means for performing the operations, such as various hardware and/or software component(s), circuits and/or module(s). The software may include an ordered list of executable instructions to implement logical functions, and may be used by an instruction execution system apparatus or device (e.g., single or multi-core processor, a system including a processor) or be implemented in a processor readable medium.

Steps, blocks, or functions of a method or an algorithm described below may be directly implemented as hardware, a software module executed by a processor, or a combination thereof. When implemented in software, the functions may be stored as one or more instructions or code on a tangible, non-transitory computer readable medium. The software module may be stored in a storage medium including a random access memory (RAM) device, a flash memory device, a read only memory (ROM) device, an electrically programmable ROM (EPROM) device, an electrically erasable programmable ROM (EEPROM) device, registers, a hard disk, a removable disk, a compact disc (CD) ROM device, etc.

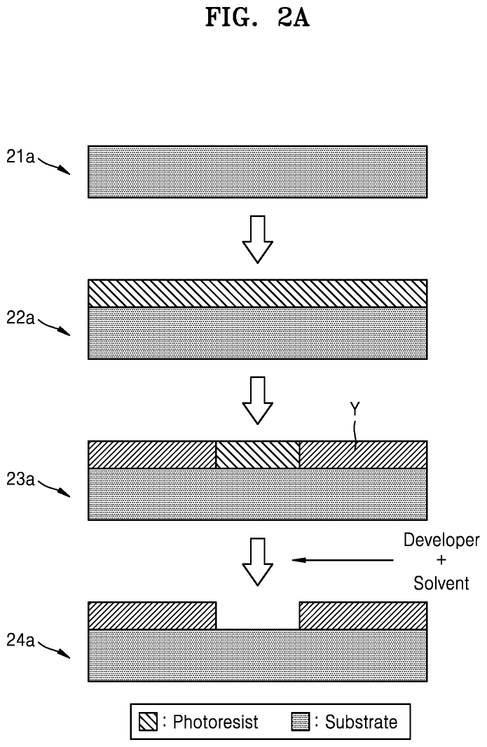

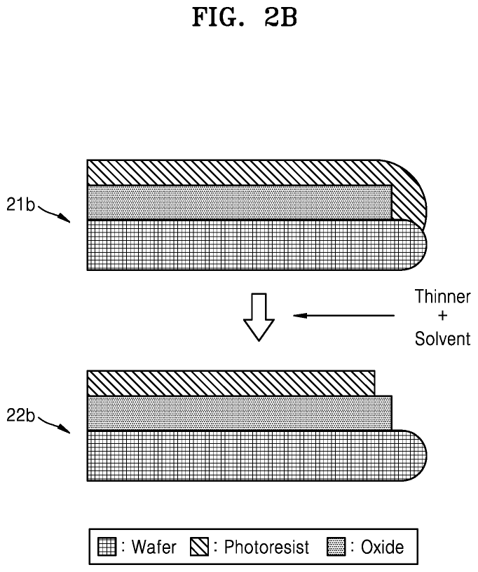

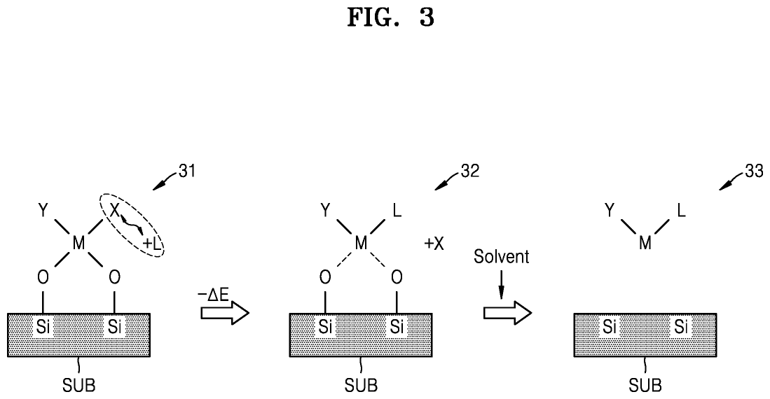

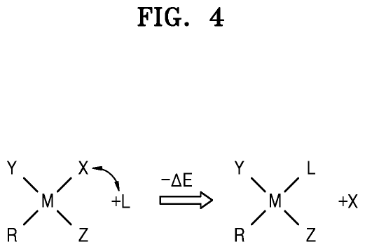

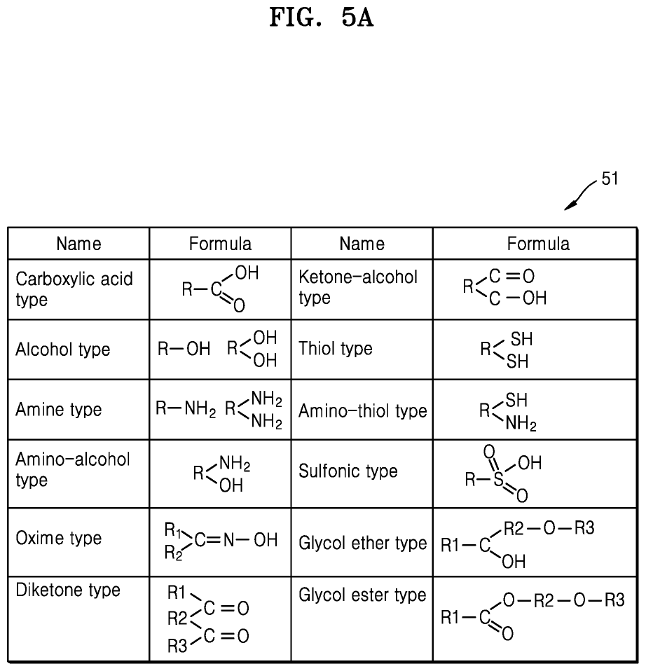

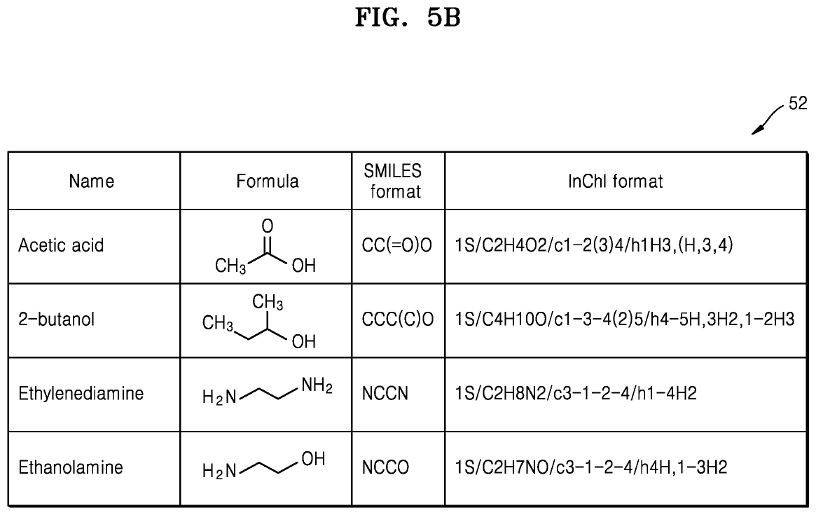

Referring to FIG. 1, input data IN may be obtained in operation S20. In some embodiments, the input data IN may define a ligand material. For example, as described hereinafter with reference to FIGS. 3 and 4, a metal-ligand complex (also referred to as metal-complex) photoresist may be used, and a metal complex photoresist may be removed based on a ligand exchange reaction. The photoresist dissolvent may include a ligand that is exchanged with a photoresist ligand, and the input data IN may include information on the ligand of the photoresist dissolvent. The input data IN may have an arbitrary format representing the ligand. Examples of the photoresist dissolvent will be described later with reference to FIGS. 2A and 2B, and examples of the input data IN will be described later with reference to FIGS. 5A and 5B.

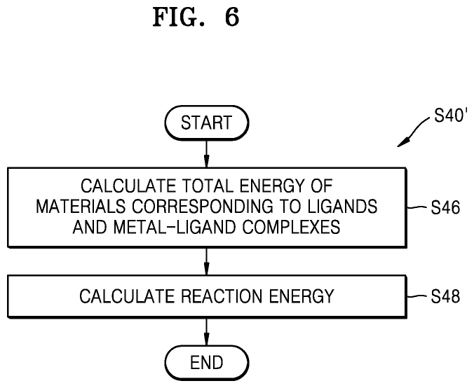

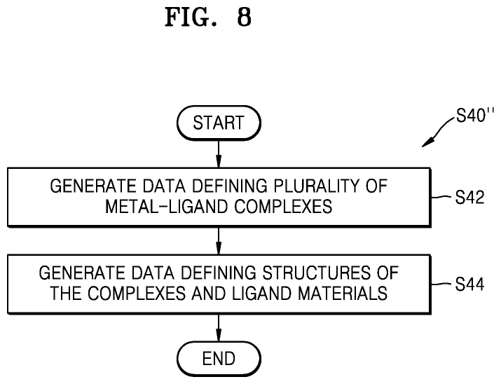

In operation S40, the reaction energy in the ligand exchange reaction may be estimated. In some embodiments, the reaction energy generated in the ligand exchange reaction may function as an index or descriptor of suitability of the photoresist dissolvent. For example, the lower the reaction energy of the ligand exchange reaction, the more easily the ligand is exchanged, and accordingly, the photoresist may be more easily eliminated. As described above, the photoresist may include a metal complex having a first metal and a first ligand. The photoresist dissolvent may include a second ligand, and thus the reaction energy may be estimated in a ligand exchange reaction in which the first ligand of the photoresist is exchanged with the second ligand of the photoresist dissolvent. Examples of operation S40 will be described later with reference to FIGS. 6 and 8.

In operation S60, a concentration of residuals of metal (referred to as residual concentration) may be estimated. As will be described later with reference to FIG. 3, after the ligand exchange reaction, the metal complex of the photoresist is dissolved into the photoresist dissolvent by exchanging the first ligand with the second ligand, so that the photoresist may be removed by the photoresist dissolvent. For example, as described above, the residual concentration of the first metal may be estimated in the ligand exchange reaction in which the first ligand of the metal-complex is exchanged with the second ligand. The lower the residual concentration of a metal, the higher the compatibility of the photoresist dissolvent with respect to the photoresist, and the higher the performance of photolithography. That is, the residual concentration of the metal may indicate the result of photolithography and may indicate the practical performance of the photoresist dissolvent.

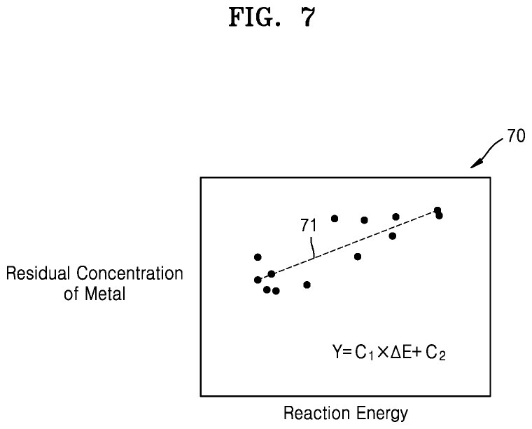

In some embodiments, the residual concentration of metal may be estimated based on a physical model (PM). The physical model PM may define a relationship between the reaction energy of the ligand exchange reaction and the residual concentration of the metal, and the residual concentration of the metal corresponding to the estimated reaction energy in operation S40 may be estimated based on the physical model PM. Due to the physical model PM using the residual concentration of the metal, the photoresist dissolvent may be practically evaluated. An example of the physical model PM will be described later with reference to FIG. 7.

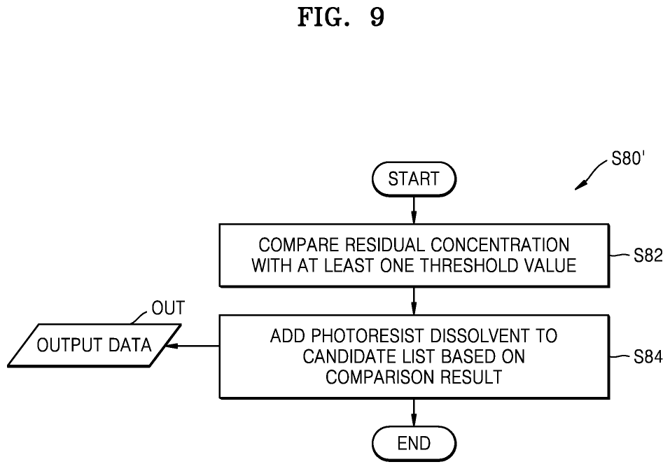

In operation S80, the photoresist dissolvent may be verified. For example, the photoresist dissolvent may be verified based on the residual concentration of the metal estimated in operation S60. In some embodiments, the residual concentration of metal may be compared to at least one threshold value and verified based on the comparison result. As shown in FIG. 1, output data OUT may be generated by verifying the photoresist dissolvent. In some embodiments, the output data OUT may include the reaction energy and the residual concentration of the metal corresponding to a photoresist dissolvent. In some embodiments, the output data OUT may include a candidate list, and the candidate list may include information on the photoresist dissolvents determined to be available by verification. An example of operation S80 will be described later with reference to FIG. 9.

Discovering a photoresist dissolvent suitable for a photoresist usually requires numerous repeated experiments on multiple combinations between various light sources, various photoresists, and various photoresist dissolvents; however, numerous repeated experiments may be difficult to perform in practice. However, as described above, the reaction energy of the ligand exchange reaction and the residual concentration of the metal may be estimated, and the photoresist dissolvent may be quickly and accurately verified by the estimated reaction energy and the residual concentration of the metal, and thus the photoresist dissolvent corresponding to the photoresist may be easily discovered. In addition, as the photoresist dissolvent is easily discovered, the photolithography process may be easily designed even though the light source and/or the photoresist are/is changed according to the photolithography process.

In some embodiments, an integrated circuit may be manufactured in a semiconductor manufacturing process subsequent to operation S80. For example, the integrated circuit may be manufactured by patterning a plurality of layers using at least one mask. The semiconductor manufacturing process may include a series of sub-processes. For example, a front-end-of-line (FEOL) process of the semiconductor manufacturing process may include, for example, a process of planarization and cleaning of a wafer, a process of forming a trench, a process of forming a well, a process of forming a gate electrode, and a process of forming a source and a drain, and individual elements of a semiconductor device, such as transistors, capacitors, resistors, etc., may be formed on a substrate by the FEOL process. In addition, a back-end-of-line (BEOL) process of the semiconductor manufacturing process may include, for example, a process of silicidation of the gate, source, and drain areas, a process of adding a dielectric, a planarization process, a process of forming a hole, a process of adding a metal layer, a process of forming a via, a process of forming a passivation layer, etc., and the individual elements such as transistors, capacitors, resistors, etc. may be interconnected with one another by the BEOL process. In some embodiments, a middle-of-line (MOL) process may be performed between the FEOL process and the BEOL process, and thus various contacts may be formed on the individual elements. Then, the integrated circuit (IC) device may be packaged in a semiconductor package and used as a component in various applications.

The semiconductor manufacturing process may include a patterning process in which the photoresist dissolvent that has passed the verification in operation S80 is used. As described above, an optimal photoresist dissolvent for a photoresist may be discovered, and the integrated circuit may be accurately manufactured by the semiconductor manufacturing process as designed, and as a result, the performance and yield of the integrated circuit may be improved.

FIGS. 2A and 2B are diagrams illustrating processing steps for manufacturing an integrated circuit in the semiconductor manufacturing process according to an embodiment. For example, FIGS. 2A and 2B include cross-sectional views of structures sequentially formed in the semiconductor manufacturing process. The integrated circuit may include an analog signal processing circuit, a digital signal processing circuit, and a combination thereof.

The semiconductor manufacturing process may include various sub-processes for forming various patterns in the integrated circuit. For example, the semiconductor manufacturing process may include a plurality of photolithography processes. In this regard, a photolithography process refers to a process of forming a pattern by transferring a geometric pattern of a photomask to a photosensitive chemical photoresist by using light. The photoresist may include a positive photoresist in which a portion to which light is irradiated is dissolved by a developer and a negative photoresist in which a portion to which light is not irradiated is dissolved by a developer. FIG. 2A shows exemplary processing steps for forming a photoresist pattern in a photolithography process using a negative photoresist.

Referring to FIG. 2A, a substrate may be provided in a first state 21a. For example, a plurality of patterns may be formed on the substrate by at least one sub-process.

In a second state 22a, the negative photoresist may be applied on the substrate. As shown in FIG. 2A, a material for the negative photoresist may be dissolved in a first solvent as a solute, and a solution of the material and the first solvent may be applied on the substrate, to thereby form a negative photoresist layer on the substrate. In some embodiments, the negative photoresist layer may be applied on an oxide layer by a spin coating process. Particularly, the photoresist may include an inorganic material that is smaller than an organic polymer and has a higher light absorption rate so as to form a more detailed pattern. For example, the photoresist may include organometallics, such as a metal-ligand complex consisting of a metal atom M and organic molecular R, X and Y ligands, or metal-oxygen clusters with the formula MₙOₘRᵢXⱼYₖ where R, X and Y are organic molecular ligands and n, m, i, j, k>1. In some embodiments, after the negative photoresist layer is applied on the substrate, the substrate may be heated, so that the excess solvent may be removed from the photoresist layer.

In the second state 22a, a photomask may be aligned over the substrate, and light may be irradiated to the aligned photomask. Light having a shorter wavelength, such as extreme ultraviolet (EUV), may be used for forming a more detailed photoresist pattern. In a third state 23a, as shown in FIG. 2A, a portion of the negative photoresist layer exposed to the light passing through the photo mask may be chemically transformed, and thus the light-exposed portion of the photoresist layer may be formed into a material Y different from the photoresist.

Thereafter, a developing process may be performed on the substrate which experienced light exposure by using a developer, and a portion of the photoresist layer to which the light was not irradiated may be dissolved by the developer in the third state 23a. Then, in a fourth state 24a, a solvent may be provided and the dissolved photoresist may be removed by the solvent. The developing process refers to a process of removing a portion of the photoresist layer chemically modified by light or photoresist using a developer. In some embodiments, subsequent processes, such as etching and cleaning, may be performed after the fourth state 24a.

Referring to FIG. 2B, a stack structure may be provided on a wafer in such a configuration that an oxide layer and a photoresist layer may be sequentially formed on the wafer in a first state 21b. For example, the oxide layer may be formed on the wafer, and the photoresist layer may be formed on the oxide layer. As shown in FIG. 2B, when forming the photoresist layer, the photoresist may be applied on a side of the oxide layer at an edge of the wafer. The photoresist on the side of the oxide layer may be referred to as an edge bead, which may be required to be removed as a defect. In the first state 21b, a thinner may be provided, and a portion of the photoresist layer exposed to the thinner may be dissolved. Then, in a second state 22b, a solvent may be provided, and the dissolved photoresist may be removed by the solvent.

The developer described above with reference to FIG. 2A and the thinner described above with reference to FIG. 2B may be required to dissolve the photoresist significantly, and both the developer and the thinner may be referred to herein as a photoresist dissolvent. Accordingly, the photoresist and the photoresist dissolvent may be important for forming an accurate photoresist pattern and/or for accurately removing the edge bead. Here, the photoresist and the photoresist dissolvent may be determined by estimating the reaction energy of a ligand exchange reaction and the residual concentration of a metal, as described above with reference to FIG. 1. Accordingly, the photoresist and the photoresist dissolvent may be accurately and easily determined.

FIG. 3 is a diagram illustrating an operation of the photoresist dissolvent according to an embodiment. As described above with reference to the figures, the photoresist dissolvent may be used for removing the dissolved photoresist.

Referring to FIG. 3, in a first state 31, a photoresist layer may be formed on the substrate SUB. The substrate SUB may include silicon (Si), and the photoresist layer may include a metal-ligand complex. The metal-ligand complex may include a metal atom M, and may include a plurality of oxygen atoms O and organic molecules X and Y bonded to the metal atom M. In some embodiments, the metal atom M may include tin (Sn), antimony (Sb), hafnium (Hf), zirconium (Zr), titanium (Ti), cobalt (Co), iron (Fe), chromium (Cr), palladium (Pd), platinum (Pt), zinc (Zn), and aluminum (Al), as non-limiting examples. Herein, among the organic molecules X and Y bonded to the metal atom M of the photoresist, the organic molecule X, which is exchanged with an organic molecule L of the photoresist dissolvent described later, is referred to as first ligand, and the organic molecule L, which is bonded to the metal atom M by exchanging with the organic molecule X, is referred to as second ligand.

In a second state 32, the first ligand may be exchanged with the second ligand. For example, as shown in FIG. 3, the photoresist dissolvent, such as the developer in FIG. 2A or the thinner in FIG. 2B, may include the organic molecule L, and the organic molecule L of the photoresist dissolvent may be bonded to the metal atom M in place of the organic molecule X of the metal-ligand complex in the photoresist together with a release of the reaction energy ΔE in a first state 31, so that the energy of the second state 32 may be lower than that of the first state 31 by as much as the reaction energy ΔE. As shown by the dotted line in FIG. 3, the bond of the metal atom M and the oxygen atom O may be weakened in the second state 32 from in the first state 31 due to the organic molecule L bonded to the metal atom M.

A solvent may be provided in the second state 32, and accordingly, the photoresist including metal atom M and organic molecules Y and L may be removed from the substrate SUB in a third state 33. When the ligand exchange reaction is actively performed, that is, when the first ligand is actively exchanged with the second ligand, the metal-ligand complex may be removed from the substrate SUB, and thus, the substrate SUB may have a low residual concentration of the metal in the third state 33. Otherwise, when the ligand exchange reaction is inactively performed, that is, when the first ligand is not actively exchanged with the second ligand, the metal-ligand complex may remain on the substrate SUB, and thus, the residual concentration of the metal may be high on the substrate SUB in the third state 33. That is, the function of the photoresist dissolvent may be modeled as a ligand exchange reaction between the photoresist and the photoresist dissolvent, and the performance of the photoresist dissolvent may depend on the ligand exchange reaction.

FIG. 4 is a reaction scheme illustrating a ligand exchange reaction model according to an embodiment. As described above with reference to FIG. 3, the photoresist may be removed by a photoresist dissolvent based on a ligand exchange reaction.

Referring to FIG. 4, the metal-complex may include a metal atom M and organic molecules R, X, Y, and Z, and the organic molecules R, X, Y, and Z may be ligands of the metal atoms M. The metal-complex may react with the organic molecule L, and the organic molecule X (that is, a first ligand) of the metal-complex may be exchanged with the organic molecule L (that is, a second ligand). Accordingly, the metal-complex may include a metal atom M and organic molecules R, Y, Z, and L, and the organic molecule X may be separated from the metal-ligand complex. The reaction energy ΔE of the ligand exchange reaction and the residual concentration of the metal described above with reference to FIG. 3 may be used to verify the photoresist dissolvent containing organic molecules L. That is, the lower the reaction energy ΔE is, the more active the ligand exchange reaction may be, and thus, it is advantageous that the photoresist dissolvent includes the second ligand in a manner in which the reaction energy ΔE is as low as possible.

FIGS. 5A and 5B are tables showing examples of the input data IN according to an embodiment. As described above with reference to FIG. 1, the input data IN may define a ligand material. For example, FIG. 5A shows first data 51 defining a plurality of organic molecules, and FIG. 5B shows second data 52 defining chemical structures of ligands. In some embodiments, the input data IN in FIG. 1 may include the first data 51 shown in the table of FIG. 5A and the second data 52 shown in the table of FIG. 5B. In some embodiments, the input data IN in FIG. 1 may further include data not shown in FIGS. 5A and 5B.

Referring to FIG. 5A, the first data 51 may define a plurality of organic molecules. In some embodiments, the first data 51 may include 12 types of organic molecules. For example, as shown in FIG. 5A, the first data 51 may define a carboxylic acid type, an alcohol type, an amine type, an amino-alcohol type, an oxime type, a diketone type, a ketone-alcohol type, a thiol type, an amino-thiol type, a sulfonic type, a glycol ether type, and a glycol ester type. It is noted that the types of organic molecules defined by the first data 51 are not limited to those listed in the table of FIG. 5A. In some embodiments, the chemical formula of FIG. 5A may be omitted from the first data 51.

Referring to FIG. 5B, the second data 52 may define chemical structures of a plurality of ligands. In some embodiments, second data 52 may define chemical structures of 4 ligands. For example, as shown in FIG. 5B, the second data 52 may define acetic acid, 2-butanol, ethylenediamine, and ethanolamine.

In some embodiments, the second data 52 may include a string including a series of characters defining the chemical structure of a ligand. For example, the second data 52 may include a string expressed based on a simplified molecular-input line-entry system (SMILE) code, a SMILES arbitrary target specification (SMARTS) code, and an international chemical identifier (InChI). FIG. 5B shows the second data 52 including the character strings expressed based on SMILES and InChI. For example, the acetic acid may be expressed by a string ‘CC(═O)O’ or ‘1S/C2H4O2/c1-2(3)4/h1H3,(H,3,4)’. The 2-butanol may be expressed as ‘CCC(C)O’ or ‘1S/C4H10O/c1-3-4(2)5/h4-5H,3H2,1-2H3’. The ethylenediamine may be expressed as ‘NCCN’ or ‘1S/C2H8N2/c3-1-2-4/h1-4H2’. The ethanolamine may be expressed as ‘NCCO’ or ‘1S/C2H7NO/c3-1-2-4/h4H,1-3H2’. In some embodiments, the second data 52 may include strings only based on SMILES or InChI. In some embodiments, the second data 52 may further include strings in an additional format, such as based on SMARTS. In some embodiments, the chemical formula in FIG. 5B may be omitted from the second data 52.

FIG. 6 is a flowchart illustrating an aspect of a method of discovering a photoresist dissolvent, according to an embodiment. For example, the flow chart in FIG. 6 shows an example embodiment of operation S40 in FIG. 1. As described above with reference to FIG. 1, the reaction energy of the ligand exchange reaction may be estimated in operation S40″ in FIG. 6. As shown in FIG. 6, operation S40′ may include operation S46 and operation S48. Hereinafter, the method of discovering the photoresist dissolvent is described with reference to FIG. 6 together with FIG. 4.

Referring to FIG. 6, in operation S46, the total energy of each of materials corresponding to the ligands and the metal-ligand complexes may be calculated. For example, the total energy of a metal-ligand complex (referred to as a first complex) including a metal atom M and an organic molecule X (that is, a first ligand), the total energy of an organic molecule L, the total energy of a metal-ligand complex (referred to as a second complex) including the metal atom M and an organic molecule L (that is, a second ligand), and the total energy of organic molecule X may be each calculated.

In some embodiments, the total energy of each of the materials corresponding to the ligands and the metal-ligand complexes may be calculated based on a first-principles simulation (or a first principle calculation). For example, the total energy may be calculated based on a molecular orbital theory-based method, such as the Hartree-Fock method, the semi-empirical quantum chemistry method, Moller-Plesset perturbation method, the coupled cluster method, and the quantum Monte Carlo method, as non-limiting examples. The total energy may also be calculated based on a density function theory-based method, such as the Thomas-Fermi model method, the orbital-free density functional theory method, the linearized augmented plane-wave method, and the projected augmented wave method, as non-limiting examples.

In operation S48, the reaction energy may be calculated. In some embodiments, the reaction energy may be calculated by the difference between the sum of the total energy prior to the ligand exchange reaction and the sum of the total energy after the ligand exchange reaction. For example, the reaction energy ΔE may be calculated by the following [Equation 1].

ΔE=E(M−L)+E(X)−(E(M−X)+E(L)) [Equation 1]

In [Equation 1], E(M-L) denotes the total energy of the second complex, which is the metal-ligand complex including the metal atom M and the organic molecule L (the second ligand), E(X) denotes the total energy of the organic molecule X, E(M-X) denotes the total energy of the first complex, which is the metal-ligand complex including the metal atom M and the organic molecule X (the first ligand), and E(L) denotes the total energy of the organic molecule L.

FIG. 7 is a graph illustrating a physical model PM according to an embodiment. As described above with reference to FIG. 1, the physical model PM may be used to estimate the residual concentration of metal from the reaction energy of the ligand exchange reaction.

Referring to FIG. 7, the horizontal axis of a graph 70 represents the reaction energy and the vertical axis of the graph 70 represents the residual concentration of metal in a logarithmic scale. The graph 70 shows an experimental relationship of the reaction energy and the residual concentration of metal for some pairs of the photoresist and the photoresist dissolvent. As shown in FIG. 7, as the reaction energy decreases, the residual concentration of metal generally decreases, and the physical model PM may be defined based on the graph 70.

In some embodiments, the physical model PM may include a linear model. For example, as shown in FIG. 7, a trend line 71 may be derived from the points of the graph 70, and the physical model PM may be defined as in [Equation 2] below.

Y=C₁×ΔE+C₂ [Equation 2]

In [Equation 2], Y denotes the residual concentration of the metal in a logarithmic scale, and C₁ and C₂ denote constants derived from the trend line 71. Accordingly, when the reaction energy ΔE of the ligand exchange reaction is estimated, the residual concentration of the metal corresponding to the reaction energy ΔE may be estimated, and the photoresist dissolvent may be verified.

FIG. 8 is a flowchart showing another aspect of a method of discovering a photoresist dissolvent according to an embodiment. For example, the flowchart in FIG. 8 shows an example embodiment of operation S40 in FIG. 1. As described above with reference to FIG. 1, the reaction energy of the ligand exchange reaction may be estimated in operation S40″ in FIG. 8. As shown in FIG. 8, operation S40″ may include operation S42 and S44. In some embodiments, operation S40 in FIG. 1 may include operations S42 and S44 in FIG. 8 as well as operations S46 and S48 in FIG. 6, where operations S42 and S44 may be performed before operations S46 and S48. Hereinafter, the method of discovering the photoresist dissolvent is described with reference to FIG. 8 together with FIGS. 5A and 5B.

Referring to FIG. 8, data defining a plurality of metal-ligand complexes may be generated in operation S42. In some embodiments, metals and ligands may be combined based on the input data IN, and metal-complexes may be defined according to the combinations of the metal and the ligand. In order to find the optimal pair of photoresist and photoresist dissolvent, various metal-ligand complexes that are included in the photoresist or that occur after the photoresist dissolvent is applied to the photoresist may be defined. The metal-ligand complexes may be defined based on the input data IN, for example, the first data 51 of FIG. 5A and the second data 52 of FIG. 5B, and thus, a plurality of actual metal-complexes may be defined. Herein, the data for defining a plurality of complexes may be referred to as third data.

In operation S44, data defining the structures of the complex and the ligand materials may be generated. For example, the structures of the complexes defined in operation S46 may be defined, the structures of materials of the ligands in the complexes, that is, the material structures of the first ligands may be defined, and the structures of materials (referred to as second materials) with which the first ligands are exchanged may be defined. The data defined in operation S44 may be used for calculating the total energy in operation S46 in FIG. 6. Herein, the data for defining the structures of the complexes and the ligand materials may be referred to as fourth data.

FIG. 9 is a flowchart showing still another aspect of a method of discovering a photoresist dissolvent according to an embodiment. For example, the flowchart in FIG. 9 shows an example embodiment of operation S80 in FIG. 1. As described above with reference to FIG. 1, the photoresist dissolvent may be verified in operation S80′ in FIG. 9. As shown in FIG. 9, operation S80′ may include operations S82 and S84. Hereinafter, the method of discovering the photoresist dissolvent is described with reference to FIG. 9 together with FIG. 1.

Referring to FIG. 9, the residual concentration may be compared with at least one threshold value in operation S82. In some embodiments, at least one threshold value may be preset, and at least one threshold value may correspond to at least one value of the residual concentration of metal. Two or more ranges of the residual concentration of metal may be defined by at least one threshold value, and the concentration range to which the residual concentration of metal belongs may be identified by comparing the residual concentration of metal with at least one threshold value.

In operation S84, a photoresist dissolvent may be added to the candidate list based on the comparison result. For example, when the residual concentration is within a first range in operation S82, the photoresist dissolvent may be determined to have passed the verification and may be added to the candidate list of the output data OUT. When the residual concentration is within a second range in operation S82, the photoresist dissolvent may be determined to have not passed the verification, and the photoresist dissolvent may not be added to the candidate list.

FIG. 10 is a graph illustrating an example of the concentration range defined by the threshold value, according to an embodiment. As described above with reference to FIG. 9, the residual concentration of metal may be compared with at least one threshold value, and a photoresist dissolvent may be added to the candidate list according to the comparison result.

In some embodiments, a threshold value THR may be predefined. For example, as shown in FIG. 10, the threshold value THR may correspond to a specific value of the residual concentration of metal. Thus, the concentration range may be defined into a first range R1 below the threshold value THR and a second range R2 above the threshold value THR. As described above with reference to FIG. 7, the physical model PM may define a relationship in which the reaction energy ΔE and the residual concentration of metal are proportional to each other. Accordingly, when the residual concentration of metal is within the first range R1, the photoresist dissolvent corresponding to the residual concentration of metal may be determined to have passed the verification and may be added to the candidate list. When the residual concentration of metal is within the second range R2, the photoresist dissolvent corresponding to the residual concentration of metal may be determined to have not passed the verification and may not be added to the candidate list. In some embodiments, two or more threshold values may be predefined, and two or more candidate lists, each having different priorities, may be defined, respectively.

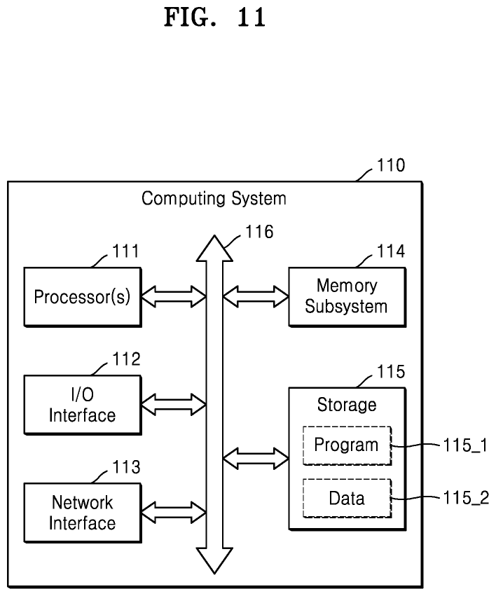

FIG. 11 is a block diagram showing a computing system 110, according to an embodiment. In some embodiments, the method of discovering the photoresist dissolvent, described above with reference to the figures, may be performed in the computing system 110 in FIG. 11.

The computing system 110 may include a stationary computing system, such as a desktop computer, a workstation, or a server, and a portable computing system, such as a laptop computer. As shown in FIG. 11, the computing system 110 may include at least one processor 111, an input/output interface 112, a network interface 113, a memory subsystem 114, a storage 115, and a bus 116, and the processor 111, the input/output interface 112, the network interface 113, the memory subsystem 114, and the storage 115 may communicate with one another via the bus 116.

The at least one processor 111 may be referred to as at least one processing unit, and execute a program like CPU, GPU, NPU, or DSP. For example, at least one processor 111 may have access to the memory subsystem 114 via the bus 116 and may execute various instructions stored in the memory subsystem 114. In some embodiments, the computing system 110 may further include an accelerator as a dedicated hardware for performing a specific function at high speed.

The input/output interface 112 may include an input device such as a keyboard or a pointing device, and/or an output device such as a display device or a printer, or may provide access to an input device and/or an output device. The user may trigger the execution of the program 115_1 and/or the loading of the data 115_2 by the input/output interface 112, and the input data IN in FIG. 1 and the output data OUT in FIG. 1 may also be input/output by the input/output interface 112.

The network interface 113 may provide access to a network outside the computing system 110. For example, the network may include multiple computing systems and communication links, and the communication links may include wired links, optical links, wireless links, and any other type of links.

The memory subsystem 114 may store a program 115_1 or at least a portion thereof for a method of estimating the solubility described above with reference to the drawings, and at least one processor 111 may perform at least some of the steps included in the method of estimating the solubility by executing the program (or instructions) stored in the memory subsystem 114. The memory subsystem 114 may include a read only memory (ROM) device, a random access memory (RAM) device, etc.

The storage 115 may include a non-transitory storage medium, and the data 115_2 stored in the storage 115 may not be lost even if the power supplied to the computing system 110 is cut off. For example, the storage 115 may include a non-volatile memory device, or a storage medium such as a magnetic tape, an optical disk, and a magnetic disk. In addition, the storage 115 may be detachable from the computing system 110. As shown in FIG. 11, the program 115_1 and the data 115_2 may be stored in the storage 115. At least a portion of the program 115_1 may be loaded into the memory subsystem 114 before being executed by at least one processor 111. In some embodiments, the storage 115 may store a file written in a programming language, and the program 115_1 or at least a portion thereof, which is generated from the file by a compiler, etc., may be loaded into the memory subsystem 114. At least one processor 111 may perform at least a portion of the method of discovering the photoresist dissolvent described above with reference to the drawings by executing the program 115_1. The data 115_2 may include data required for performing the method for discovering a photoresist dissolvent described above with reference to the drawings, for example, the input data IN and/or the physical model PM in FIG. 1. In addition, the data 115_2 may include data generated in performing the method for discovering a photoresist dissolvent described above with reference to the drawings, for example, the output data OUT in FIG. 1.

As described above, exemplary embodiments have been disclosed in the drawings and the specification. Although the embodiments have been described by using specific terms in this specification, they are only used for the purpose of explaining the technical idea of the present disclosure, and are not used to limit the scope of the present disclosure described in the claims. Therefore, those of ordinary skill in the art will understand that various modifications and other equivalent embodiments are possible therefrom. Therefore, the true technical scope of protection of the present disclosure should be determined by the technical idea of the appended claims.

While example embodiments of the disclosure have been particularly shown and described, it will be understood that various changes in form and details may be made therein without departing from the spirit and scope of the following claims.

1. A method comprising:
obtaining input data defining a ligand material;
estimating a reaction energy of a ligand exchange reaction based on the input data, a first ligand of a first complex comprising a first metal and the first ligand being exchanged with a second ligand in the ligand exchange reaction;
estimating a residual concentration of the first metal corresponding to the reaction energy, based on a physical model; and
verifying a photoresist dissolvent providing the second ligand based on the residual concentration.

2. The method of claim 1, wherein the estimating the reaction energy comprises:
determining a total energy of a second complex comprising the first metal and the second ligand, a total energy of a second material corresponding to the second ligand, a total energy of the first complex, and a total energy of a first material corresponding to the first ligand; and
determining the reaction energy based on the total energy of the second complex, the total energy of the second material, the total energy of the first complex, and the total energy of the first material.

3. The method of claim 2, wherein the reaction energy is determined as an energy difference between a sum of the total energy of the second complex and the total energy of the first material and a sum of the total energy of the first complex and the total energy of the second material.

4. The method of claim 2, wherein each of the total energy of the second complex, the total energy of the second material, the total energy of the first complex, and the total energy of the first material is determined based on a first-principles simulation.

5. The method of claim 2, wherein the physical model is a linear model in which the residual concentration of the first metal is in a logarithmic scale.

6. The method of claim 1, wherein the input data comprises:
first data defining a plurality of organic molecules; and
second data defining chemical structures of a plurality of ligands.

7. The method of claim 6, wherein the estimating the reaction energy comprises:
generating third data defining a plurality of complexes comprising the first complex by combining metals and ligands based on the first data and the second data; and
generating fourth data defining structures of the first complex, a first material of the first ligand, and a second material of the second ligand, based on the first data and the second data.

8. The method of claim 1, wherein the verifying the photoresist dissolvent comprises:
comparing the residual concentration with at least one threshold; and
adding the photoresist dissolvent to a candidate list based on a result of the comparing.

9. The method of claim 1, further comprising:
manufacturing an integrated circuit in a semiconductor manufacturing process, wherein the semiconductor manufacturing process comprises a patterning process using the photoresist dissolvent passing the verifying.

10. A system comprising:
a non-transitory storage medium storing instructions; and
at least one processor configured to execute the instructions to:
obtain input data defining a ligand material;
estimate a reaction energy of a ligand exchange reaction based on the input data, a first ligand of a first complex comprising a first metal and the first ligand being exchanged with a second ligand in the ligand exchange reaction;
estimate a residual concentration of the first metal corresponding to the reaction energy based on a physical model; and
verify a photoresist dissolvent providing the second ligand based on the residual concentration.

11. The system of claim 10, wherein the at least one processor is further configured to execute the instructions to estimate the reaction energy by:
determine a total energy of a second complex comprising the first metal and the second ligand, a total energy of a second material corresponding to the second ligand, a total energy of the first complex, and a total energy of a first material corresponding to the first ligand; and
determine the reaction energy based on the total energy of the second complex, the total energy of the second material, the total energy of the first complex, and the total energy of the first material.

12. The system of claim 11, wherein the at least one processor is further configured to execute the instructions to determine the reaction energy as an energy difference between a sum of the total energy of the second complex and the total energy of the first material and a sum of the total energy of the first complex and the total energy of the second material.

13. The system of claim 11, wherein the total energy of the second complex, the total energy of the second material, the total energy of the first complex, and the total energy of the first material are each calculated based on a first-principles simulation.

14. The system of claim 11, wherein the physical model is a linear model in which the residual concentration of the first metal is in a logarithmic scale.

15. The system of claim 10, wherein the input data comprises:
first data defining a plurality of organic molecules; and
second data defining chemical structures of a plurality of ligands.

16. The system of claim 15, wherein the at least one processor is further configured to execute the instructions to estimate the reaction energy by:
generating third data defining a plurality of complexes comprising the first complex by combining metals and ligands based on the first data and the second data; and
generating fourth data defining structures of the first complex, a first material of the first ligand, and a second material of the second ligand, based on the first data and the second data.

17. The system of claim 10, wherein the at least one processor is further configured to execute the instructions to verify the photoresist dissolvent by:
comparing the residual concentration with at least one threshold; and
adding the photoresist dissolvent to a candidate list based on a result of the comparing.

18. A non-transitory storage medium storing instructions executed by at least one processor to perform a method comprising:
obtaining input data defining a ligand material;
estimating a reaction energy of a ligand exchange reaction based on the input data, a first ligand of a first complex comprising a first metal and the first ligand being exchanged with a second ligand in the ligand exchange reaction;
estimating a residual concentration of the first metal corresponding to the reaction energy based on a physical model; and
verifying a photoresist dissolvent providing the second ligand based on the residual concentration.

19. The non-transitory storage medium of claim 18, wherein the estimating the reaction energy comprises:
determining a total energy of a second complex comprising the first metal and the second ligand, a total energy of a second material corresponding to the second ligand, a total energy of the first complex, and a total energy of a first material corresponding to the first ligand; and
determining the reaction energy based on the total energy of the second complex, the total energy of the second material, the total energy of the first complex, and the total energy of the first material.

20. The non-transitory storage medium of claim 19, wherein the reaction energy is determining as an energy difference between a sum of the total energy of the second complex and the total energy of the first material and a sum of the total energy of the first complex and the total energy of the second material.

21-25. (canceled)
